(19) United States
(12) Patent Application Publication
(10) Pub. No.: US 2007/0005023 A1
(43) Pub. Date: Jan. 4, 2007

(54) Safety device for IV set

(76) Inventors: Yu-Yueh Lin, Taipei (TW); Kun-Jung Yang, Jungli City (TW)

Correspondence Address: LOWE HAUPTMAN BERNER, LLP, 1700 DIAGONAL ROAD, SUITE 300, ALEXANDRIA, VA, 22314, US

(21) Appl. No.: 11/153,358

(22) Filed: Jun. 16, 2005

(51) Int. Cl. A61M 5/00

(52) U.S. Cl. 604/250

(57) ABSTRACT

A safety device for IV (intravenous) set includes a removable tubing disposed on an IV set. Before use, the removable tuning is clipped on a duct of the IV set, near a needle. After use, the removable tubing is operated for moving from the rear end of the needle (needle bed) to the front end (needle tubing) thereof so that the front end of the removable tubing extends forward and covers the needle tubing completely. While the rear end of the removable tubing is locked and fixed on the needle bed. Therefore, the removable tubing is easy and safe to use and the needle tubing retracts completely into the removable tubing so as to achieve the safety requirement of the use of the needle.

### BACKGROUND OF THE INVENTION

The present invention relates to a safety device for IV (intravenous) set, especially to a removable tubing that is operated easily and safely so as to make a needle tubing retract and being fixed inside the removable tubing for preventing exposing and needle injuries.

Refer to FIG. 1, an I.V. set 1 is a tube used to connect an infusion bag 2 with a patient for long-term drug infusion. It is one of the most popular use of mass-produced medical devices. A conventional IV set consists of a tube 10, a drip chamber 11, a roller clamp 12 and a needle having a needle bed 13 and a needle tubing 14. Before being used, the needle is enclosed with thin tubing 15 so as to avoid stab wounds. For the sake of safety and sanitation, general IV sets are disposed after use. However, there is no safety device arranged on the conventional IV set 1. Thus the needle tubing 14 is exposed after use and easy to have needle stick injuries. There is danger as well as panic of infection. Although the original thin tubing 15 can be used to isolate the used needle tubing 14, it's still a bit difficult and dangerous operation for people to insert the needle tubing 14 into the thin tubing 15. Therefore, there is no safety devices on conventional IV set 1 and safety issues have been raised for users.

### SUMMARY OF THE INVENTION

Therefore it is a primary object of the present invention to provide a safety device for IV set that uses a removable tubing disposed thereof. After being used, the removable tubing moves from the rear end (needle bed) towards the front end (needle tubing) and covers the needle tubing completely while a tail end of the removable tubing is locked and fixed on the needle bed. Thus the removable tubing with features of easy operation and safety requirement achieves similar effect of safety needle.

It is another object of the present invention to provide a safety device for IV set that has a removable tubing on a IV set. The removable tuning is with simple structure and is disposable together with the IV set after being used. Due to simple structure, the device can be mass-produced.

It is a further object of the present invention to provide a safety device for IV set that has a removable tubing on a IV set. The removable tubing can be clipped temporarily on the duct of the IV set, near the needle while it has no effect on the use of the IV set 1. The device is easy to use and is convenient for users.

### BRIEF DESCRIPTION OF THE DRAWINGS

FIG. 1 is a schematic drawing of a conventional IV set being used;

FIG. 2 is a perspective view of the present invention before being used;

FIG. 3 is an explosive view of a removable tubing in accordance with the present invention;

FIG. 4 is a schematic drawing of the removable tubing before being used in accordance with the present invention;

FIG. 5 is a schematic drawing of the removable tubing after use in accordance with the present invention;

FIG. 6 is a schematic drawing of the IV set after use in accordance with the present invention;

FIG. 7 is a lateral sectional view of the removable tubing before being used in accordance with the present invention;

FIG. 8 is a schematic drawing of the removable tubing shown in FIG. 7 being used in accordance with the present invention;

FIG. 9 is a schematic drawing of the removable tubing shown in FIG. 7 after use in accordance with the present invention;

FIG. 10 is an explosive view of a double-tubular removable tubing of another embodiment in accordance with the present invention;

FIG. 11 is a lateral sectional view of the removable tubing shown in FIG. 10 before being used in accordance with the present invention;

FIG. 12 is a schematic drawing of the removable tubing shown in FIG. 11 being used in accordance with the present invention;

FIG. 13 is a schematic drawing of the removable tubing shown in FIG. 11 after use in accordance with the present invention;

FIG. 14 is an explosive view of a single-tubular removable tubing of another embodiment in accordance with the present invention;

FIG. 15 is a lateral sectional view of the removable tubing shown in FIG. 14 before being used in accordance with the present invention;

FIG. 16 is a is a schematic drawing of the removable tubing shown in FIG. 15 after use in accordance with the present invention.

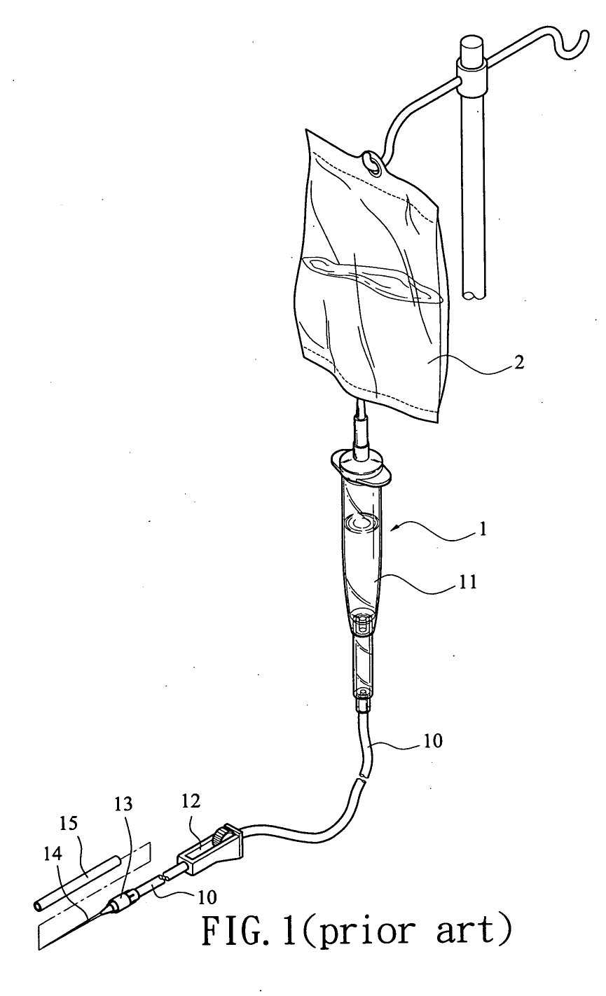

### DETAILED DESCRIPTION OF THE PREFFERED EMBODIMENT

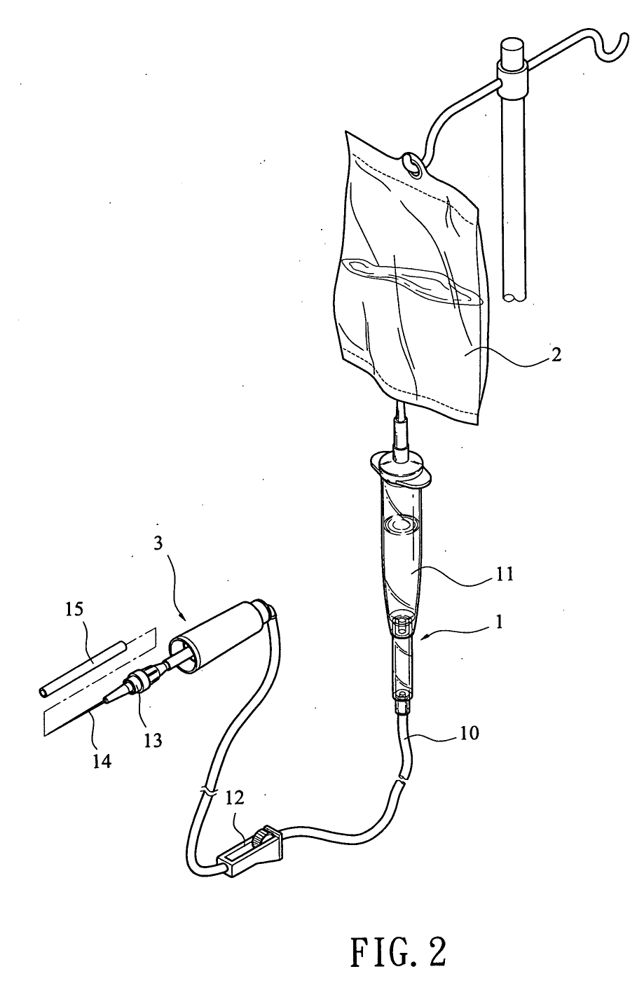

A safety device of an IV set in accordance with the present invention includes a removable tuning 3 disposed on an IV set 1. Refer to FIG. 2, the IV set 1 is composed by a tube 10, a drip chamber 11, a roller clamp 12 and a needle having a needle bed 13 as well as a needle tubing 14. Before being used, the needle is enclosed by a thin tubing 15 that covers on the needle tubing 14. The removable tubing 3 is a tubing or conic tubing, assembled with the IV set 1. Before use of the IV set 1, the removable tubing 3 is clipped on the tube 10, near the needle bed 13 of the IV set 1.

After use of the IV set 1, users can manipulate the removable tubing 3 to move from the rear end (needle bed 13) towards the front end (needle tubing 14) so as to make the front end of the removable tuning 3 covers the needle tubing 14 completely while the tail end of the removable tuning 3 is locked on the needle bed 13 for fixation. The needle tubing 14 is retracted and located into the removable tuning 3 so as to achieve safe use of needles. The removable tuning 3 is double-tubular or single-tubular.

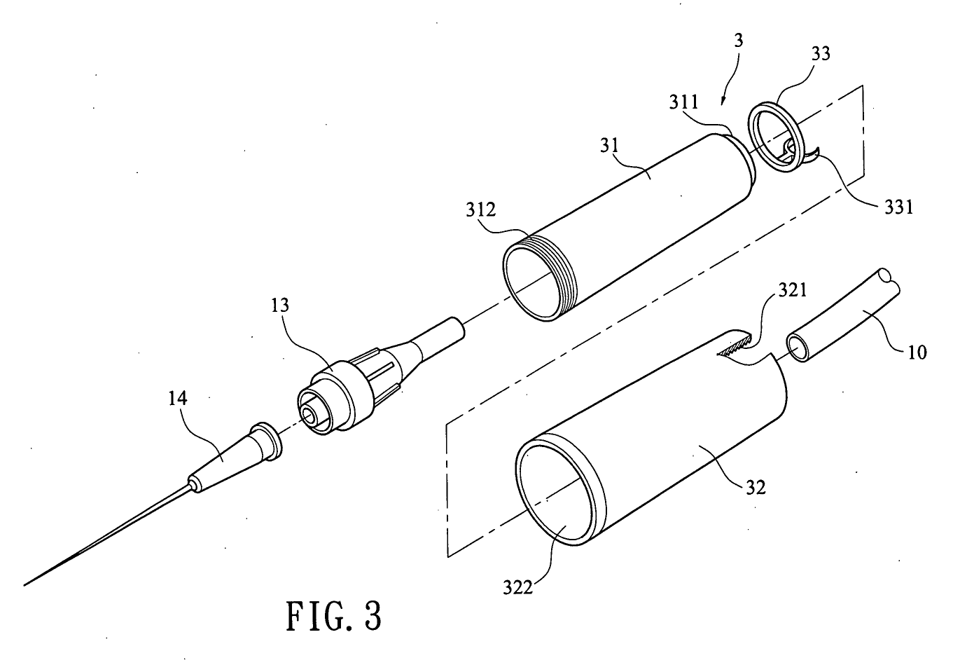

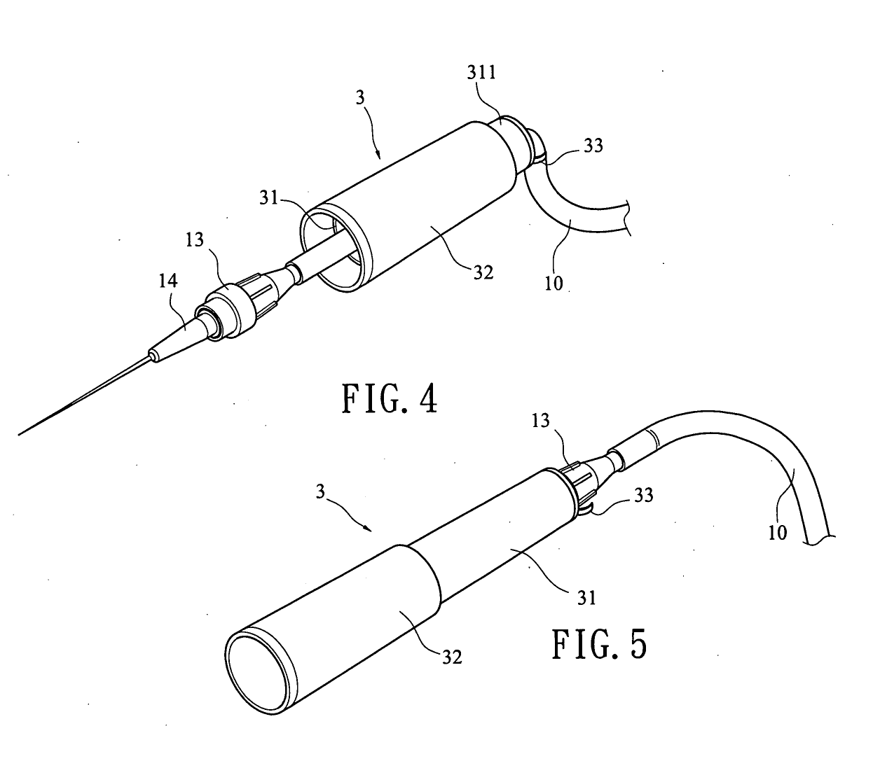

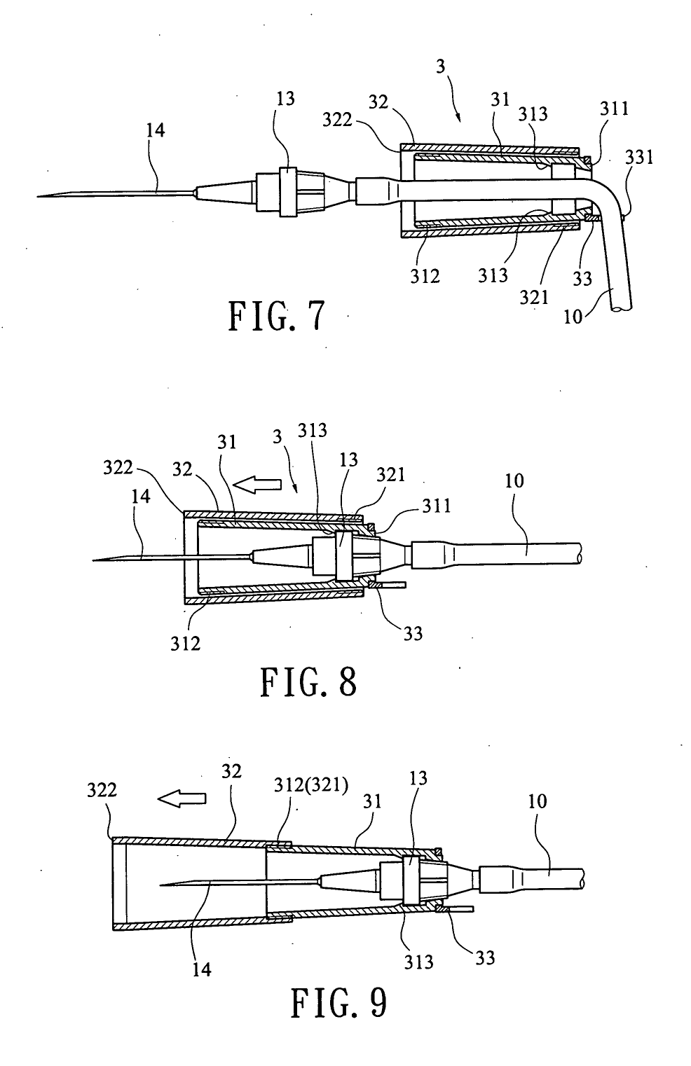

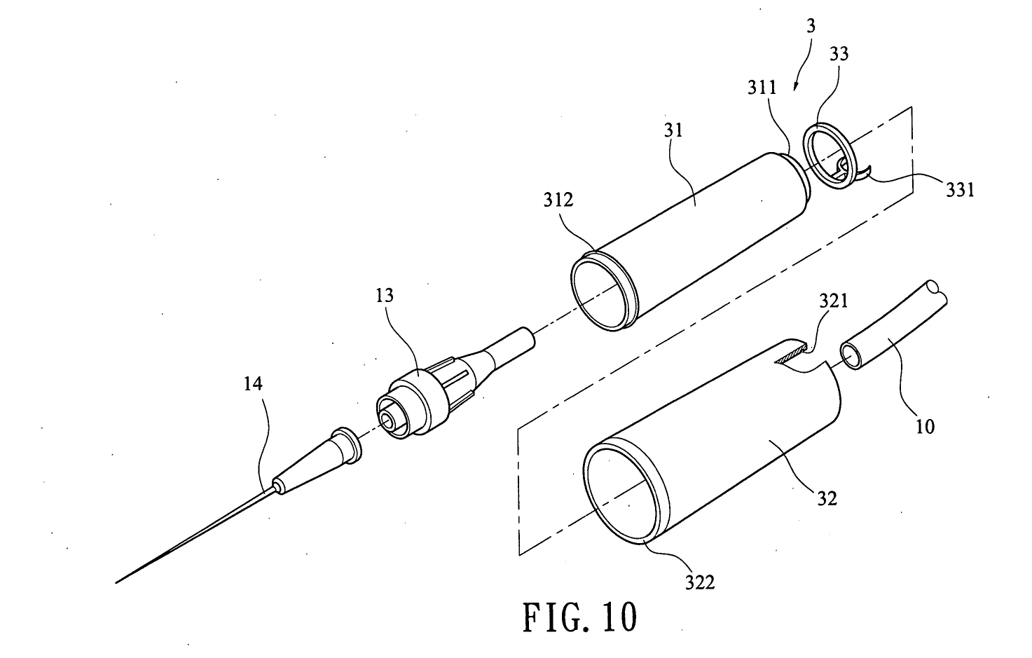

Refer from FIG. 2 to FIG. 13, the removable tubing 3 is double-tubular that encloses the duct 10. The removable tubing 3 consists of an inner conic tubing 31 and an outer conic tubing 32. The outer conic tubing 32 is inserted into the inner conic tubing 31 from the tail 311 thereof and slides therein. The diameter of the tail 311 is smaller than that of the inner conic tubing 31 so as to lock the needle bed 13 for preventing the needle bed 13 from sliding out of the tail 311. A fixing part 312 is disposed on outer surface of the front end of the inner conic tubing 31. The fixing part 312 can be an outer thread shown in FIG. 3, FIG. 7, FIG. 8, & FIG. 9, or a projective ring shown from FIG. 10 to FIG. 13. A fixing part 321 corresponding to the fixing part 312 is arranged on inner side of the rear end of the outer conic tubing 32 while it can be an inner thread shown in FIG. 3, FIG. 7, FIG. 8, & FIG. 9, or a circular slot shown from FIG. 10 to FIG. 13. When the outer conic tubing 32 moves forward, the fixing part 321 on the rear end is locked with the fixing part 312 on front end of the inner conic tubing 31 so that the head 322 of the outer conic tubing 32 extends into a longer tubing, as shown in FIG. 5. Moreover, refer to FIG. 7, a circular projection 313 is disposed near inner side of the tail 311 of the inner conic tubing 31. The front end of the circular projection 313 is a slope so that the needle bed 13 slides through the front end of the circular projection 313, then is locked between the circular projection 313 and the tail 311. In addition, a ring 33 with an elastic clip 331 is arranged on outer surface of the tail 311 of the inner conic tubing 31. The semicircular elastic clip 331 is easy to operate for clipping the removable tubing 3 on the duct 10 at the position users need.

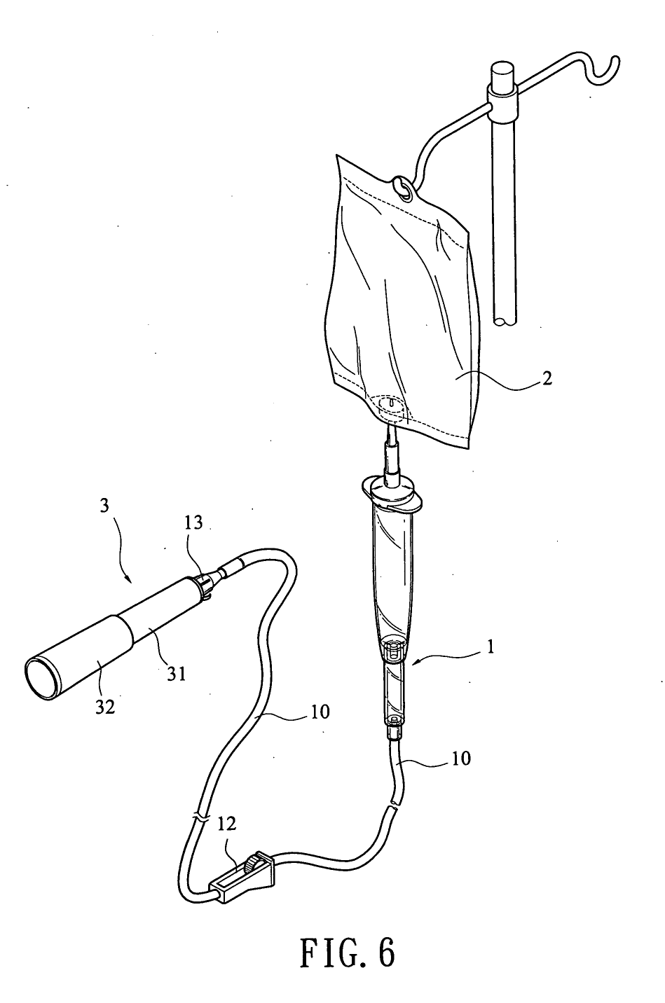

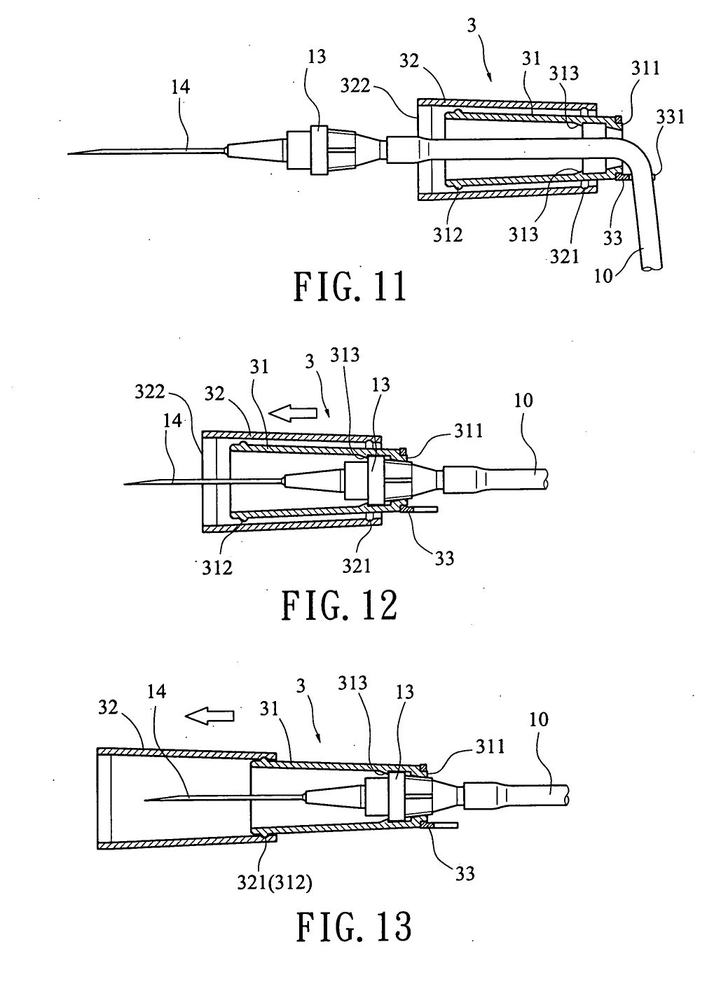

Before use, the removable tubing 3 is clipped on the duct 10 of the IV set 1, near the needle bed 13, as shown in FIG. 2 FIG. 7 & FIG. 11, without any effect on the normal use of the IV set 1. After use, before disposing, the elastic clip 331 is removed so that the removable tubing 3 is moveable. Then push the removable tubing 3 to move from the needle bed 13 (rear end) to the needle tubing 14 (the tip). During the process, the needle bed 13 is locked and fixed between the circular projection 313 and the tail 311 of the inner conic tubing 31, as shown in FIG. 8 & FIG. 12. At this moment, the outer conic tubing 32 moves forwards and then the fixing part 321 on the rear end thereof is locked with the fixing part 312 on front end of the inner conic tubing 31. Thus the head 322 of the outer conic tubing 32 extends into a tubing with enough length to cover the needle tuning 14 completely, as shown in FIG. 9 or FIG. 13. During the process, both the inner conic tubing 31 and the outer conic tubing 32 move from the needle bed 13 (rear end) to the needle tubing 14 (the tip) and then are fixed so as to make the needle tubing 14 retract completely inside the removable tubing 3, without the danger of exposure outside. Therefore, the removable tubing 3 is easy and safe to operate so as to achieve the purpose of safe use of needle, as shown in FIG. 6.

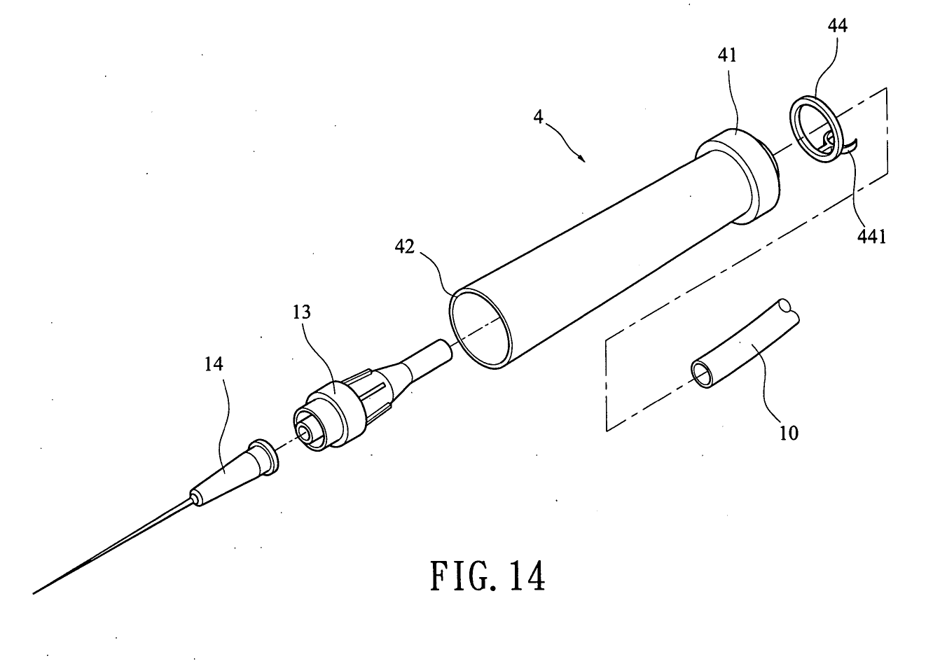

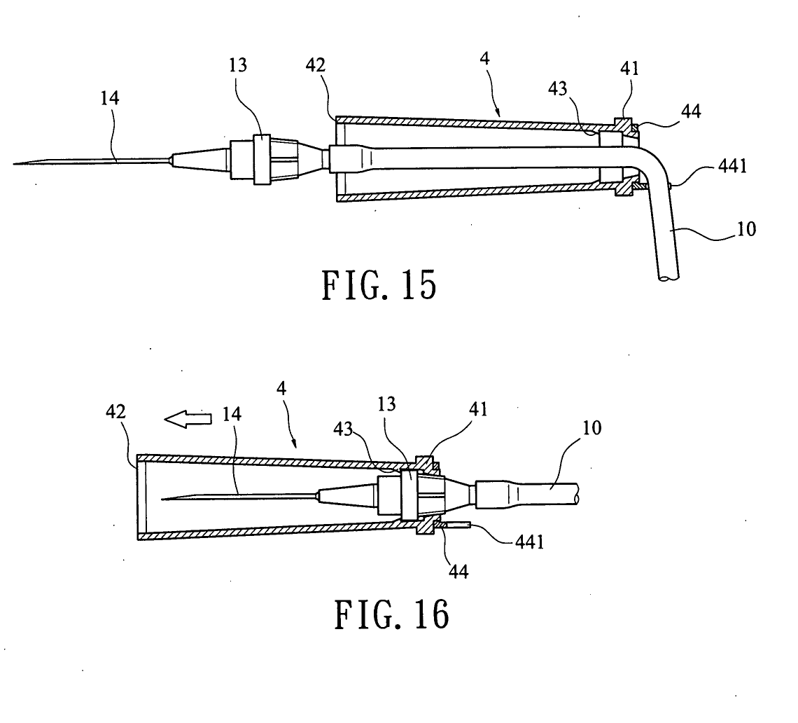

Refer to FIG. 14, FIG. 15, & FIG. 16, the removable tubing 4 is a single tube with certain length and is covered on the duct 10 of IV set. The tail 41 of the removable tubing 4 has smaller diameter so as to be locked with the needle bed 13 for fixing the needle bed 13 and preventing it from falling out of the tail 41. The length between the tail 41 and a head 42 of the removable tubing 4 is longer than that of the needle (length of the needle bed 13 plus the length of the needle tubing 14). A circular projection 43 is disposed on inner side of the removable tubing 4, near the tail 41. The front end of the circular projection 43 is a slope so that the needle bed 13 slides through the front end thereof and then hooks with the circular projection 43. The needle bed 13 is locked and fixed between the circular projection 43 and the tail 41. Furthermore, a ring 44 with an elastic clip 441 is set on the outer surface of the tail 41 and is assembled with or removed from the duct 10 by the circular elastic clip 441. The diameter of the elastic clip 441 matches the diameter of the duct 10.

Before use, the removable tubing 4 is clipped on the duct 10 of the IV set 1 by the elastic clip 441 of the ring 44, near the needle bed 13 without effect on normal use of the IV set 1, as shown in FIG. 15. After use, the removable tubing 4 is moveable by removing of the elastic clip 441. Then the removable tubing 4 slides from the needle bed 13 (rear end) to the needle tubing 14 (the tip) while the need bed 13 is fixed automatically between the circular projection 43 and the tail 41 thereof. Now the head 42 of the removable tubing 4 extends forward and covers the needle tubing 14 completely, as shown in FIG. 16. Therefore, the removable tubing 4 is easy and safe to use.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A safety device for IV (intravenous) set comprising an IV set and a removable tubing, wherein
the IV set including a tube, a drip chamber, a roller clamp and a needle having a needle bed as well as a needle tubing;
the removable tubing enclosing the duct of the IV set; after use, the removable tubing is moved from the needle bed (rear end) to the needle tubing (front end) so as to make the front end of the removable tubing extend forward and cover the needle tubing while the rear end of the removable tubing is locked and fixed on the needle bed, thereby the needle tubing retracts and is fixed inside the removable tubing.

2. The device as claimed in claim 1, wherein the removable tubing is clipped on the duct of the IV set, near the needle bed without effect on use of the IV set.

3. The device as claimed in claim 1, wherein the removable tubing is double-tubular, having an inner conic tubing and an outer conic tubing.

4. The device as claimed in claim 3, wherein the removable tubing having an inner conic tubing and an outer conic tubing while the outer conic tubing encloses and slides outside the inner conic tubing;
a rear end of the inner conic tubing having a smaller diameter for locking with the needle bed and preventing the needle bed from sliding out of the rear end thereof; a fixing part is arranged on outer surface of front end of the inner conic tubing and another fixing part corresponding to the fixing part on the inner conic tubing is arranged on inner surface of rear end of the outer conic tubing so that the outer conic tubing moves forward, assembles and locks with the inner conic tubing by the fixing parts for extending forward and covering the needle tubing.

5. The device as claimed in claim 4, wherein a circular projection is disposed on inner side of the removable tubing, near the rear end; the front end of the circular projection is a slope so that the needle bed slides through the circular projection and hooks between the circular projection and the rear end of the inner conic tube.

6. The device as claimed in claim 4, wherein a ring with an elastic clip is disposed on outer surface of the rear end of the removable tubing; the removable tubing is clipped on or removed from the duct of the IV set by the elastic clip.

7. The device as claimed in claim 4, wherein the fixing parts on outer surface of front end of the inner conic tubing and on inner surface of rear end of the outer conic tubing are various types, corresponding to each other.

8. The device as claimed in claim 4, wherein the fixing parts on outer surface of front end of the inner conic tubing and on inner surface of rear end of the outer conic tubing are outer thread and corresponding inner thread.

9. The device as claimed in claim 4, wherein the fixing parts on outer surface of front end of the inner conic tubing and on inner surface of rear end of the outer conic tubing are projective ring and corresponding slot.

10. The device as claimed in claim 1, wherein the removable tubing is single-tubular.

11. The device as claimed in claim 10, wherein the removable tubing is clipped on the duct of the IV set, near the needle bed without effect on use of the IV set.

12. The device as claimed in claim 10, wherein length of the removable tubing is longer than the length of the needle.

13. The device as claimed in claim 10, wherein the rear end of the removable tubing having a smaller diameter for locking the removable tubing on the needle bed and preventing the needle bed from sliding out of the removable tubing.

14. The device as claimed in claim 10, wherein a circular projection is disposed on inner side of the removable tubing, near the rear end; the front end of the circular projection is a slope so that the needle bed slides through the circular projection and hooks between the circular projection and the rear end of the inner conic tube.

15. The device as claimed in claim 10, wherein a ring with an elastic clip is disposed on outer surface of the rear end of the removable tubing; the removable tubing is clipped on or removed from the duct of the IV set by the elastic clip.
